(19) United States
(12) Patent Application Publication
(10) Pub. No.: US 2016/0045678 A1
(43) Pub. Date: Feb. 18, 2016

(54) Pain Reduction Devices and Related Systems and Methods

(71) Applicant: Innova Medical Design, LLC, Plymouth, MN (US)

(72) Inventors: Rommel P. Vallero, Davis, CA (US); Timothy O'Malley, Plymouth, MN (US); Daniel Mooradian, Eagan, MN (US)

(21) Appl. No.: 14/618,656

(22) Filed: Feb. 10, 2015

Related U.S. Application Data
(60) Provisional application No. 61/937,841, filed on Feb. 10, 2014.

(51) Int. Cl. A61M 5/42; A61N 1/36; A61H 23/02; A61N 1/04

(52) U.S. Cl. CPC A61M 5/422; A61N 1/0456; A61N 1/36021; A61H 23/02

(57) ABSTRACT

The various implementations disclosed and contemplated herein relate to methods, systems, and devices for reducing or eliminating the pain of various procedures that pierce or otherwise generate pain in a patient's skin. Specific embodiments relate to devices and systems having at least two stimulation components that stimulate the target area with at least one of vibration and electrical stimulation. Some embodiments include stimulation components that are positioned on the fingers of the user, while other embodiments include a syringe-receiving component with stimulation components on flexible arms extending from the syringe-receiving component.

### CROSS-REFERENCE TO RELATED APPLICATION(S)

This application claims priority to U.S. Provisional Application 61/937,841, filed Feb. 10, 2014 and entitled Systems, Methods, and Devices for Reducing the Pain of Cosmetic Injection Procedures, which is hereby incorporated herein by reference in its entirety.

### FIELD OF THE INVENTION

The embodiments disclosed herein relate to various methods and devices for reducing or eliminating the pain of various procedures that pierce or otherwise generate pain in a patient's skin, including, but not limited to, facial injection procedures such as various cosmetic or medical injection procedures applied to the face of the patient. Alternatively, the procedures can also be applied to any other part of the body. In certain implementations, the procedures can be cosmetic procedures that include the injection of botulinum toxin, dermal fillers, and/or other cosmetic drugs for cosmetic purposes. In other embodiments, the procedures can be medical procedures that include the injection of botulinum toxin or other drugs or fluids for various medical purposes. Finally, various implementations relate to tattoo application or removal procedures. Certain embodiments relate to methods or devices that deliver stimulation in the form of vibration, electrical stimulation, or both to the patient.

### BACKGROUND OF THE INVENTION

Cosmetic injection procedures have grown in popularity over the past two decades. One of the most popular procedures involves the use of botulinum toxin, and other newer drugs that are aimed at achieving similar or enhanced results. Since the FDA announced regulatory approval in 2002 of botulinum type A (Botox Cosmetic) for the temporary treatment of frown lines on the face, the cosmetic use of botulinum toxin type A has become widespread. Minute amounts of botulinum toxin are typically injected into the face using a small needle and syringe to temporarily paralyze muscles which cause wrinkling.

Similarly, cosmetic fillers have enjoyed widespread use in recent years and are also injected into skin using a small needle and syringe in order to fill and reduce the appearance of facial wrinkles. These fillers are typically made up of hyaluronic acid and marketed under the brand names of “Restylane” and “Juvederm,” among others.

Since its introduction, botulinum toxin has also become increasingly popular for medical, non-cosmetic applications as well, including, for example, treatments for depression, migraine headaches, cerebral palsy, and urinary incontinence.

Additional painful injection-related procedures that are applied to the face and other areas of the body include tattoo application and removal procedures. These procedures include both needle injections and/or non-needle procedures such as laser-based procedures. One in five Americans have tattoos, and over 20% of those people with tattoos would like to have them removed.

Procedures like those above are commonly applied to sensitive areas of the face, lips, and other body areas and can cause significant discomfort to those electing to undergo them. In addition, multiple painful injections are usually given in a single sitting, with as many as 30 injections applied throughout the face, head, neck, and/or other sensitive areas, causing a prolonged painful experience. Once several of the procedures are done, the effects can last only a number of months, requiring recurrent uncomfortable visits to have the injections repeated. In the case of tattoo removal, sessions can total as many as 7 to 15 sessions over 3 to 24 months. Practitioners will sometimes apply ice packs to the patient's skin just before the injection in order to help reduce discomfort. This technique, however, can often be messy, cumbersome, and time consuming. Some practitioners offer topical anesthetic creams to their patients. These creams are applied to the areas to be injected but typically take from about thirty minutes to about one hour or more to be effective. This prolonged onset or repeated interruption to apply more ice has made these types of topical anesthetics difficult to incorporate within the comparatively short procedure times involved with cosmetic injections. In addition, these added procedures cause an additional level of discomfort and inconvenience for the patient. In other cases, additional painful injections of lidocane are employed to achieve a more complete pain block, thereby causing widespread numbness for several hours after the procedure.

Cosmetic injection procedures also differ from other types of medical injection procedures in that the target injection area is often the face, which has multiple, varied contours and much thinner skin than other parts of the body. This can also be true for other areas of the body as it relates to medical injections of botulinum toxin and tattoo application and removal procedures. Because of these variations, a practitioner often uses the thumb and forefinger of one hand to pinch and/or stretch the skin in order to facilitate the insertion of the needle with the other hand. This technique places the fingers in close proximity to the injection site and exposes the practitioner to accidental needle stick injury.

There is a need in the art for improved systems, methods and devices for reducing or eliminating the pain from various injection procedures.

### BRIEF SUMMARY OF THE INVENTION

Discussed herein are various embodiments relating to various methods and devices for reducing or eliminating pain of cosmetic or medical injection procedures and other similar procedures that cause pain to the skin, including the injection of botulinum toxin, the injection of cosmetic dermal fillers, the injection of other drugs or fluids, and tattoo application and removal procedures. The various embodiments include pain reduction or elimination using either electrical or vibration stimulation, or both. More specifically, certain embodiments relate to handheld devices or devices worn on the fingertips and wrist that provide pain reduction and other additional benefits using either electrical or vibration stimulation, or both.

In Example 1, a pain reduction device for reducing the pain of injection procedures and other procedures that cause skin pain comprises a first stimulation component, a second stimulation component, and a controller operably coupled to the first and second stimulation components, the controller comprising a stimulation generating module. The first stimulation component comprises a first cavity and a first stimulation element. The first cavity is defined within the first stimulation component and configured to receive at least a portion of a first distal phalanges of a first digit of a hand of a user. The first stimulation element is disposed on an outer surface of the first stimulation component. The second stimulation component comprises a second cavity and a second stimulation element. The second cavity is defined within the second stimulation component and configured to receive at least a portion of a second distal phalanges of a second digit of the hand of the user. The second stimulation element is disposed on an outer surface of the second stimulation component.

Example 2 relates to the pain reduction device according to Example 1, wherein the stimulation generating module comprises at least one of a vibration stimulation generating unit configured to transmit vibration energy to the first and second stimulation components and an electrical stimulation generating unit configured to transmit electrical energy to the first and second stimulation components.

Example 3 relates to the pain reduction device according to Example 1, wherein the first digit comprises an index finger and the second digit comprises a thumb.

Example 4 relates to the pain reduction device according to Example 1, wherein the first cavity is configured to receive the first distal phalanges and at least a portion of an intermediate phalanges of the first digit and the second cavity is configured to receive the second distal phalanges and at least a portion of an intermediate phalanges of the second digit.

Example 5 relates to the pain reduction device according to Example 1, wherein at least a portion of the outer surface of the first stimulation component comprises a first substantially curved area and at least a portion of the outer surface of the second stimulation component comprises a second substantially curved area.

Example 6 relates to the pain reduction device according to Example 5, wherein the first stimulation element is disposed on at least a portion of the first substantially curved area and the second stimulation element is disposed on at least a portion of the second substantially curved area.

Example 7 relates to the pain reduction device according to Example 1, wherein the first stimulation component comprises a first puncture resistant area and the second stimulation component comprises a second puncture resistant area.

Example 8 relates to the pain reduction device according to Example 1, wherein the first and second stimulation components are puncture resistant.

Example 9 relates to the pain reduction device according to Example 1, wherein the controller is configured to detect signals created by placing the first and second stimulation components into contact with each other in predetermined patterns, wherein the controller is configured to be triggered by the signals to alter at least one operating parameter.

Example 10 relates to the pain reduction device according to Example 1, wherein the controller is configured to be wearable on a wrist of the user.

Example 11 relates to the pain reduction device according to Example 1, wherein the controller is configured to sense contact of at least one of the first and second stimulation elements with skin of a patient, whereby the controller is configured to be triggered to actuate the stimulation generating module to generate stimulation.

Example 12 relates to the pain reduction device according to Example 1, wherein the pain reduction device comprises a glove, wherein the first stimulation component is associated with a first finger of the glove and the second stimulation component is associated with a second finger of the glove.

In Example 13, a pain reduction device for reducing the pain of injection procedures and other procedures that cause skin pain comprises a body comprising a syringe receiving cavity, a first flexible arm coupled to the body, a second flexible arm coupled to the body, and a controller operably coupled to the first and second stimulation components, the controller comprising a stimulation generating module. The first flexible arm extends distally from the body and comprises a first stimulation component disposed at a distal end of the first flexible arm. The second flexible arm extends distally from the body and comprises a second stimulation component disposed at a distal end of the second flexible arm. The first and second flexible arms are coupled to the body such that a space is defined between the first and second flexible arms.

Example 14 relates to the pain reduction device according to Example 13, wherein the body is configured to removably receive a syringe in the syringe receiving cavity such that a needle extending from the syringe is positionable between the first and second flexible arms, wherein a tip of the needle is in proximity with the first and second stimulation components.

Example 15 relates to the pain reduction device according to Example 14, wherein the first and second flexible arms are configured to flex under mild pressure, thereby allowing the needle to puncture skin in a target area while the first and second stimulation components maintain continuous contact with the skin.

In Example 16, a method for reducing the pain of injection procedures and other procedures that cause skin pain comprises positioning a first stimulation component on a first digit of a hand of a user, positioning a second stimulation component on a second digit of the hand, contacting skin of a patient with the first and second stimulation components at or near a procedure site, delivering stimulation to the skin with the first and second stimulation components, and performing a procedure on or around the skin between or proximal to the first and second stimulation components.

Example 17 relates to the method according to Example 16, wherein the positioning the first stimulation component on the first digit further comprises positioning the first stimulation component on a first distal phalanges of the first digit and the positioning the second stimulation component on the second digit further comprises positioning the second stimulation component on a second distal phalanges of the second digit.

Example 18 relates to the method according to Example 16, wherein the delivering stimulation to the skin comprises delivering at least one of vibration stimulation and electrical stimulation.

Example 19 relates to the method according to Example 16, wherein the delivering stimulation to the skin with the first and second stimulation components further comprises delivering the stimulation to the skin with a first stimulation element disposed on the first stimulation component and a second stimulation element disposed on the second stimulation component.

Example 20 relates to the method according to Example 16, further comprising protecting the first and second digits from an accidental needle stick with the first and second stimulation components, wherein the first and second stimulation components are puncture resistant.

While multiple embodiments are disclosed, still other embodiments of the present invention will become apparent to those skilled in the art from the following detailed description, which shows and describes illustrative embodiments of the invention. As will be realized, the invention is capable of modifications in various obvious aspects, all without departing from the spirit and scope of the present invention. Accordingly, the drawings and detailed description are to be regarded as illustrative in nature and not restrictive.

### BRIEF DESCRIPTION OF THE DRAWINGS

FIG. 1A is a perspective view of a combination pain reduction and needle safety device according to one embodiment.

FIG. 1B is a perspective view of a portion of the combination pain reduction and needle safety device of FIG. 1A.

FIG. 1C is a perspective cutaway view of a portion of the combination pain reduction and needle safety device of FIG. 1A.

FIG. 1D is a perspective cutaway view of a portion of the combination pain reduction and needle safety device of FIG. 1A.

FIG. 2 is a perspective view of a combination pain reduction and needle safety device according to another embodiment.

FIG. 3A is a perspective view of a combination pain reduction and needle safety device according to a further embodiment.

FIG. 3B is a perspective view of a portion of the combination pain reduction and needle safety device of FIG. 3A.

FIG. 3C is a perspective cutaway view of a portion of the combination pain reduction and needle safety device of FIG. 3A.

FIG. 4A is a perspective view of a combination pain reduction and needle safety device according to an additional embodiment.

FIG. 4B is a perspective cutaway view of a portion of the combination pain reduction and needle safety device of FIG. 4A.

FIG. 5A is a perspective view of a pain reduction device according to yet another embodiment.

FIG. 5B is an alternative perspective view of the pain reduction device of FIG. 5A.

FIG. 5C is an alternative perspective view of the pain reduction device of FIG. 5A.

FIG. 5D is a perspective view of a portion of the pain reduction device of FIG. 5A.

FIG. 5E is a perspective cutaway view of a portion of the pain reduction device of FIG. 5A.

FIG. 6A is a perspective view of a pain reduction device according to another embodiment.

FIG. 6B is an alternate perspective view of the pain reduction device of FIG. 6A.

FIG. 6C is a perspective cutaway view of a portion of the pain reduction device of FIG. 6A.

FIG. 7A is a perspective view of a pain reduction device according to a further embodiment.

FIG. 7B is a perspective view of a portion of the pain reduction device of FIG. 7A.

FIG. 7C is an alternative perspective view of a portion of the pain reduction device of FIG. 7A.

FIG. 7D is a perspective cutaway view of a portion of the pain reduction device of FIG. 7A.

FIG. 8A is a perspective view of a combination pain reduction and needle safety device according to an additional embodiment.

FIG. 8B is an alternate perspective view of the combination pain reduction and needle safety device of FIG. 8A.

FIG. 8C is a perspective cutaway view of the combination pain reduction and needle safety device of FIG. 8A.

FIG. 9A is a back view of a combination pain reduction and needle safety device in the form of a glove, according to one embodiment.

FIG. 9B is a front view of the glove of FIG. 9A.

### DETAILED DESCRIPTION

The various embodiments disclosed herein are methods and devices for reducing or eliminating the pain of various injection procedures and other similar procedures that cause pain to the skin, including the injection of botulinum toxin, the injection of cosmetic dermal fillers, the injection of other drugs and/or fluids, and tattoo procedures. These procedures can include the use of a needle or needle-less procedures such as jet injection or laser procedures. The various procedures can be for cosmetic or non-cosmetic, medical purposes. The various embodiments include pain reduction or elimination using electrical and/or vibration stimulation. More specifically, the various implementations include handheld devices or devices worn on the fingertips and wrist, or devices incorporated into gloves or other hand/finger coverings that provide pain reduction and other additional benefits.

It is understood that electrical stimulation as described herein includes, but is not limited to, transcutaneous electrical nerve stimulation (“TENS”). TENS is a noninvasive, inexpensive, and safe technique to help reduce pain. Briefly stated, the application of a low voltage, low current, mild electrical signal through electrodes placed on skin counteracts other separate nerve signals indicating pain or discomfort. Similarly, vibration therapy is a safe and effective technique to alleviate pain. U.S. Pat. No. 8,121,696, incorporated herein by reference, describes various methods of using TENS and/or vibration to reduce the pain of an injection or other procedure by delivering electrical and/or vibration stimuli near or around a needle stick.

It is further understood that the various embodiments disclosed or contemplated herein are intended to encompass any other type of procedure, including any other type of drug, chemical or compound that could be used instead of those noted above. More specifically, in addition to the various procedures discussed in further detail above and elsewhere herein, the various embodiments enclosed may also be used to reduce the pain of body piercing, acupuncture needle insertion, lumbar puncture needle insertion, subcutaneous injections, intramuscular injections, intravenous or intra-arterial injections, and the insertion of various other sharp objects into the skin, such as scalpels, lancets, suture needles, and staples. Further, the various implementations could be used to reduce the pain of any procedure that causes skin pain.

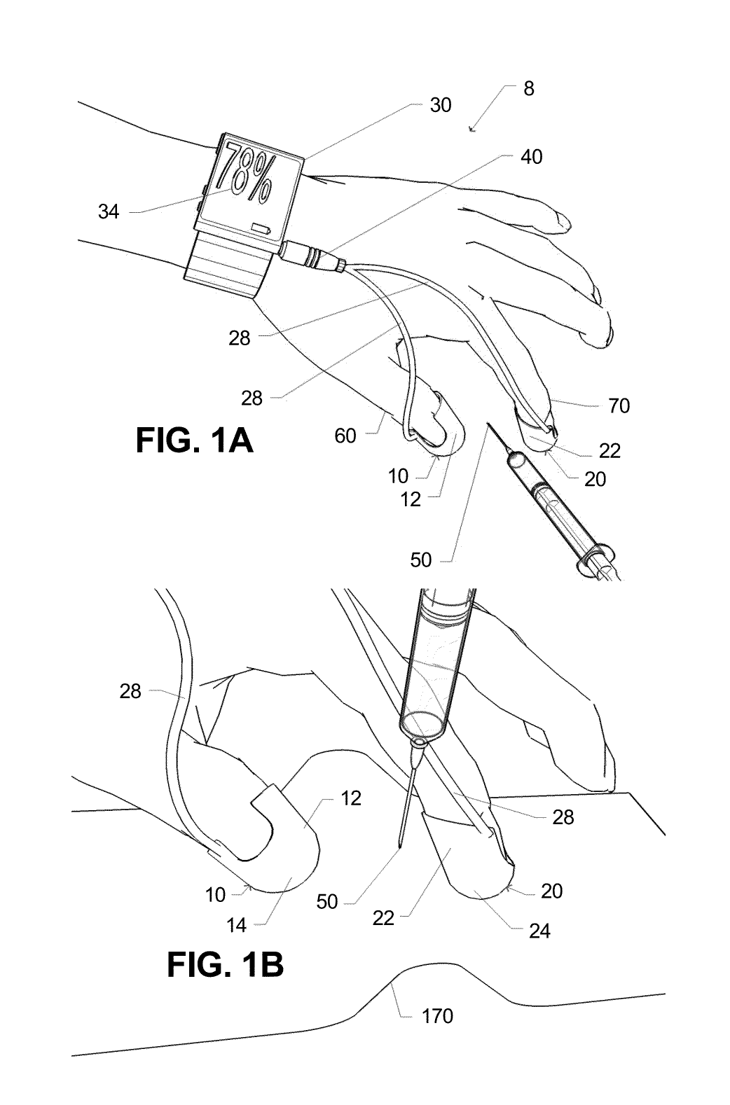

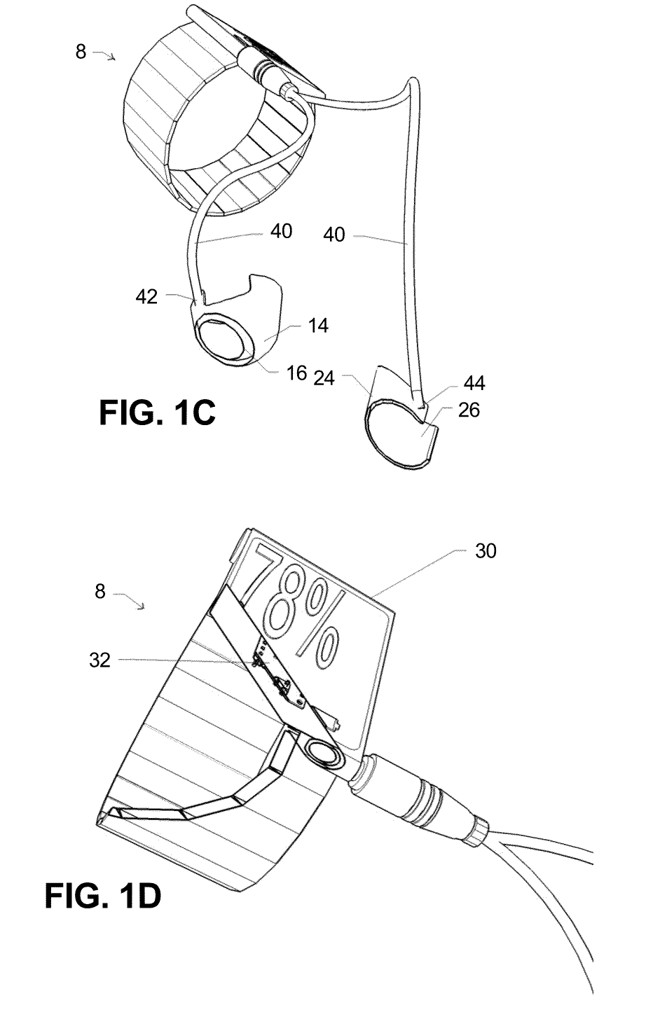

One embodiment relates to two stimulation components positioned on or over the tips of the thumb and forefinger of either the right or the left hand of the user. FIGS. 1A, 1B, 1C, and 1D depict a combination pain reduction and needle safety system 8 according to this embodiment. It consists of two stimulation components 10, 20 also referred to herein as “stimulation tip covers” or “stimulation tips.” The stimulation tip covers 10, 20 are configured to fit onto the tips of a user's fingers. In this specific example as shown, the covers 10, 20 are positioned on the thumb 60 and forefinger 70 of either the right or the left hand.

Each stimulation tip cover 10, 20 is configured to provide at least one of electrical and vibration stimulation at or near the injection or pain site. In the depicted embodiment, the covers 10, 20 are both configured to provide both electrical and vibration stimulation. Each cover 10, 20 has an outer layer (or outer portion) that is a stimulation element 14, 24. Each stimulation element 14, 24 is configured to be placed in contact with the target surface to provide the desired stimulation. In this example, the stimulation elements 14, 24 are conductive elements that are outer layers disposed around the entire exterior of the tip covers 10, 20. Alternatively, the stimulation elements 14, 24 can cover only a portion of each tip cover 10, 20. The elements 14, 24 in this implementation are electrically conductive throughout the entire outer surface of each element 14, 24, thereby maximizing contact against the varied curvature of the patient's skin 170. In certain embodiments, the contact can be further maximized by pinching skin using the cover 10, 20 in order to facilitate an injection or other procedure.

Each tip cover 10, 20 also has an inner layer 16, 26 that is made of an electrically non-conducting material such as rubber, plastic or similar material to isolate the thumb 60 and forefinger 70 from the stimulation elements 14, 24. These inner layers 16, 26 are best shown in FIG. 1C, which shows cutaway views of both tip covers 10, 20. Note that the fingers and hand in this view have been omitted for clarity.

According to one alternative embodiment, the stimulation elements 14, 24 are configured to be resistant to needle punctures, thereby functioning to help prevent accidental needle stick injury. For example, in certain implementations, the tip covers 10, 20 are meant to be worn on and around the sides of the tips of the thumb and forefinger, providing an increased level of protection against accidental needle injury. Alternatively, the elements 14, 24 have specific puncture resistant areas 12, 22 that are both electrically conductive and puncture resistant. In one embodiment, each puncture resistant area 12, 22 is made of flexible, electrically conductive carbonized rubber typical of the material used to create reusable TENS electrodes.

Both tip covers 10, 20 are electrically coupled reversibly or irreversibly to one or more wires 28, which are coupled to connectors 40 associated with a controller 30 worn on the wrist of the same hand. The wires 28 are electrically coupled to the tip covers 10, 20 at areas 42 and 44 which can best be seen in FIG. 1C.

The controller 30 has either a TENS generating component, a vibration generating component, or both. In one embodiment, the controller 30 has an optional display 34 relaying various parameters of the device. The wires 28 and connectors 40 may be typical of those used with commercially available TENS electrodes and/or commercially available vibration devices.

The TENS generating component may provide, to the stimulation elements 14, 24, specialized electrical stimulation such as a randomized waveform as described in U.S. Pat. No. 8,121,696, which is hereby incorporated herein by reference in its entirety.

The controller 30 is comprised of a microcontroller 32, best seen in FIG. 1D, which controls various aspects of the input and the output of the TENS generating component and/or vibration generating component and may also function to create the TENS stimulation and/or vibration stimulation. Typical cosmetic injection procedures and other types of procedures involve the use of both hands of the user or medical professional, thereby making it difficult for the same user to also make adjustments to the output via the controller 30. In accordance with one embodiment, the system 10 is configured to allow the practitioner to adjust the output (of electrical and/or vibrational stimulation) by touching the stimulation elements 14, 24 together in specific patterns to, for example increase or decrease the intensity of the stimulation from the vibration generating component and/or the TENS generating component. This microcontroller 32 in this implementation is configured to sense signals created by touching the stimulation elements 14, 24 together in recognized or predetermined patterns in order to affect various output of the microcontroller 32.

According to one implementation, the controller 30 also has a communication component (not shown) that allows for wireless communication between the controller 30 and an external computer or computer system. In one implementation, the communication component can be a transceiver that can communicate with a network such as the Internet or a local area network. Alternatively, the communication component can be any communication component that utilizes any wireless communication technology to transmit and receive data. In use, the communication component can be used to transmit and track patient identification, settings, including power settings, procedure data, business workflow, duration of treatment, and usage. In one specific implementation, the usage information can be used to charge a user based on that usage. In another implementation, the communication component can be used to communicate with an external database, such as, for example, a database of patient information. As such, in some embodiments, the communication component can allow for integration with a database. In further implementations, the communication component can be used to receive information relating to a specific patient and communicate that information to the controller 30. It is understood that any of the embodiments disclosed or contemplated herein can have a communication component, including the various specific embodiments described in further detail below.

In accordance with certain embodiments, one or both of the stimulation elements 14, 24 can have a microcontroller such as a microchip associated with the element 14, 24 such that the microcontroller can be used to electronically identify the stimulation element 14, 24 or limit the use of that element 14, 24. For example, in one implementation, the microcontroller in the element 14, 24 can communicate with the controller 30 to identify the element 14, 24. In further embodiments, the microcontroller in the element 14, 24 can be used to limit the use of the element 14, 24 such that the element 14, 24 can only be used for a predetermined duration or number of times before the microcontroller renders the element 14, 24 inoperable. In one embodiment, the microchip can be placed in the element 14, 24. Alternatively, the microchip can be positioned in or associated with the controller 30.

In use, as best shown in FIGS. 1A and 1B, the stimulation elements 14, 24 on the tip covers 10, 20 serve to deliver electrical and/or vibration stimulation near and around the site of injection 50 (or pain site 50) in order to reduce needle pain. These separate tip covers 10, 20 also allow the practitioner to freely manipulate the skin 170 by pinching and stretching as he/she would normally do during typical injection or treatment procedures.

As discussed above, this system 8 can be used for any number of injection or other types of procedures that cause skin pain, including the injection of botulinum toxin, the injection of cosmetic dermal fillers, the injection of other drugs and/or fluids, and tattoo procedures. These procedures can include those that include the use of a needle and those that are needle-less, such as jet injection or laser procedures. It is understood that the various procedures can include those that are cosmetic in nature and those that are non-cosmetic, medical procedures.

These tip covers are electrically coupled to a TENS generating device worn on the wrist of the same hand. The stimulation components serve to deliver electrical stimulation near and around the site of injection or other procedure. These separate tip covers also allow the practitioner to freely manipulate the skin by pinching and stretching as he/she would normally do during typical cosmetic injection procedures and other procedures. These tip covers are meant to be worn on and around the sides of the tips of the fingers, providing an increased level of protection against accidental needle injury.

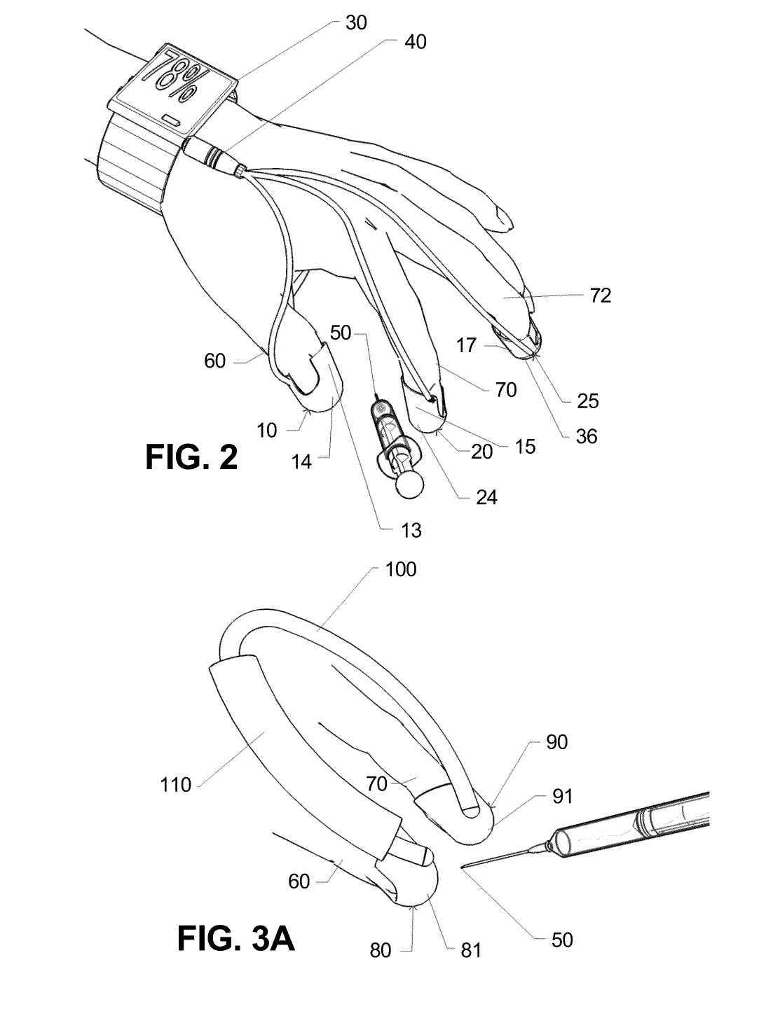

Another implementation relates to a device having three stimulation components which fit onto the tips of the thumb, forefinger, and middle finger of either the right or the left hand. FIG. 2 depicts one example of a combination pain reduction and needle safety device according to this embodiment having three tip covers 10, 20, and 25 which fit onto the tips of the thumb 60, forefinger 70, and middle finger 72 of either the right or the left hand. These stimulation components 10, 20, and 25 are similar in form, function, and construction to the stimulation components 10, 20 described in the first embodiment.

These tip covers 10, 20, and 25 have stimulation elements 14, 24, 36. According to one embodiment, the stimulation elements 14, 24, 36 can be conductive elements that are electrically conductive throughout the entire outer surface of each element 14, 24, 36, thereby maximizing contact against the varied curvature of the patient's skin. In certain embodiments, the contact can be further maximized by pinching skin using the covers 10, 20, and 25 in order to facilitate an injection or other procedure.

Each tip cover 10, 20, 25 also has an inner layer (not shown) similar to the inner layer described above that can be made of an electrically non-conducting material such as rubber, plastic or similar material to isolate the fingers from the stimulation elements 14, 24, 36.

According to one alternative implementation, the stimulation elements 14, 24, 36 are configured to be resistant to needle punctures, thereby functioning to help prevent accidental needle stick injury. Alternatively, the elements 14, 24, 36 have specific puncture resistant areas 13, 15, 17 that are both electrically conductive and puncture resistant. In one embodiment, each puncture resistant area 13, 15, 17 is made of flexible, electrically conductive carbonized rubber typical of the material used to create reusable TENS electrodes.

All three tip covers 10, 20, and 25 are electrically coupled reversibly or irreversibly to one or more wires 28, which are coupled to connectors 40 associated with a controller 30 worn on the wrist of the same hand. In one embodiment, the controller 30 is substantially similar to the controller 30 discussed above and can have generally the same components and functionality. For example, the controller 30 is comprised of a microcontroller inside the device (not shown in this figure but similar to the microcontroller 32 described above) which controls various aspects of the input and the output of the controller 30 as discussed above. Further, certain versions of this embodiment also allow for adjusting output by touching the stimulation elements 14, 24, 36 together in specific patterns to, for example increase or decrease the intensity of stimulation.

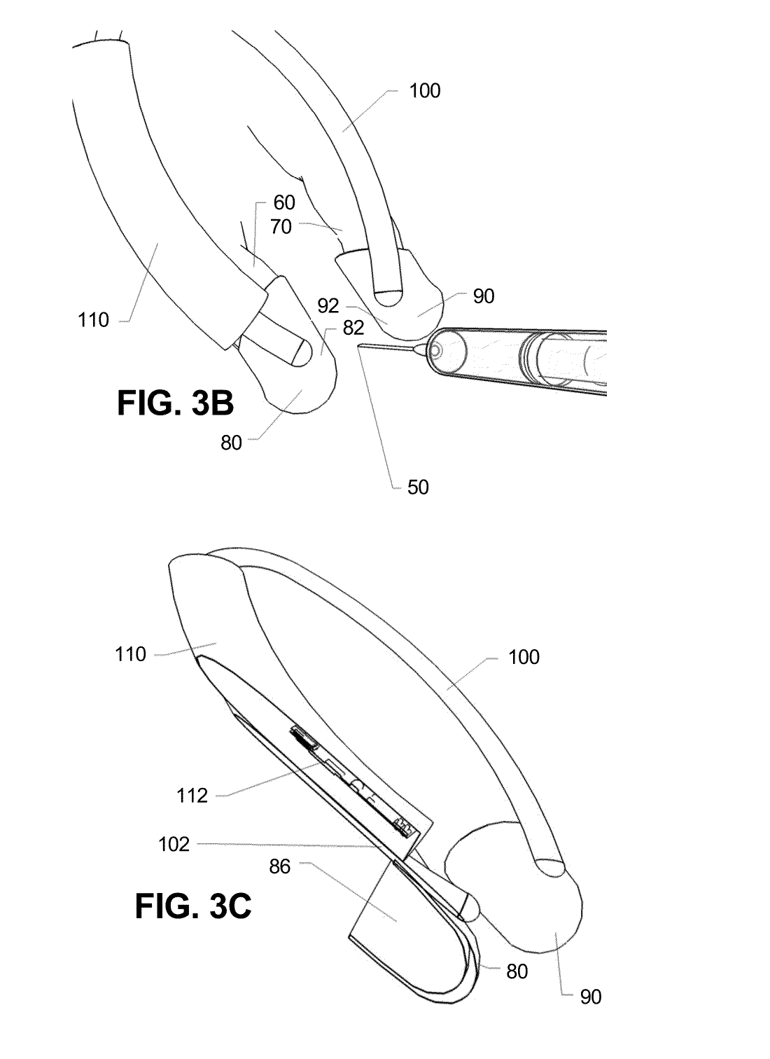

FIGS. 3A, 3B, and 3C depict a combination pain reduction and needle safety device according to a further embodiment. It consists of two stimulation components 80 and 90 which fit onto the tips of the thumb 60 and forefinger 70 of either the right or the left hand. These stimulation components 80 and 90 have outer layers that constitute stimulation elements 81, 91. According to one implementation, the stimulation elements 81, 91 are conductive elements that are electrically conductive throughout the entire outer surface of each element 81, 91, thereby maximizing contact against the varied curvature of the patient's skin.

Each tip cover 80, 90 also has an inner layer (such as inner layer 86 of cover 80 as best shown in FIG. 3C) which can be substantially similar to the inner layers of the embodiments discussed above.

In a further implementation, the stimulation elements 81, 91 can have features and components that are substantially similar to those described with respect to the embodiments above, including puncture resistance or a puncture resistance area 82, 92.

In this implementation as shown, both tip covers 80 and 90 are coupled to one another by means of a flexible coupling element 100 functioning to couple the two tip covers 80 and 90 while allowing for independent movement of the covers 80 and 90. A controller 110 is electrically coupled reversibly or irreversibly to both covers 80, 90 via wires or similar conductive material (not shown) and can couple to the device (made up of the two fingertip elements 80 and 90 and the flexible connecting element 100). The wire or other conductive material (not shown) which electrically couples the controller 110 and the two tip covers 80 and 90 is located within a hollow channel 102 defined within the flexible element 100, as best shown in FIG. 3C. The controller 110 provides to the tip covers 80 and 90 electrical and/or vibration stimulation as described above. The controller 110 is comprised of a microcontroller 112, best seen in FIG. 3C, which controls various aspects of the input and the output of controller 110 and may also function to create the TENS stimulation, vibration stimulation, or both. Like the microcontroller embodiments discussed above, this microcontroller 112 can also be configured to sense signals created by touching the conductive elements 80 and 90 together in recognized patterns in order to affect various output of the microcontroller 112.

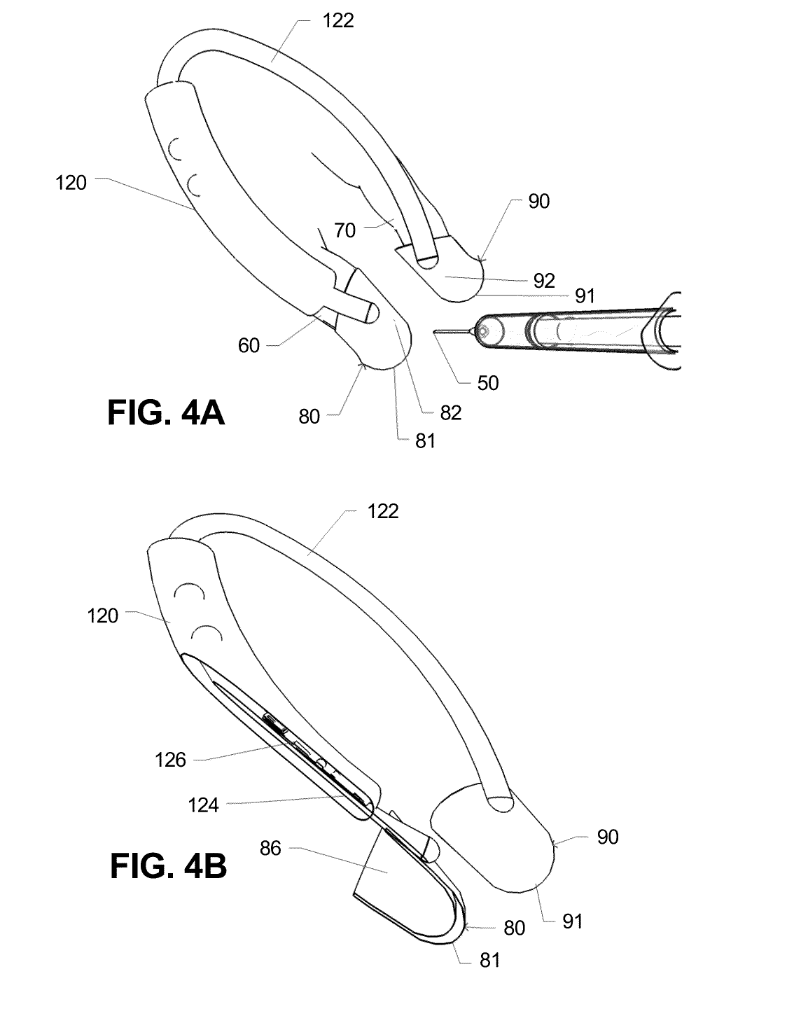

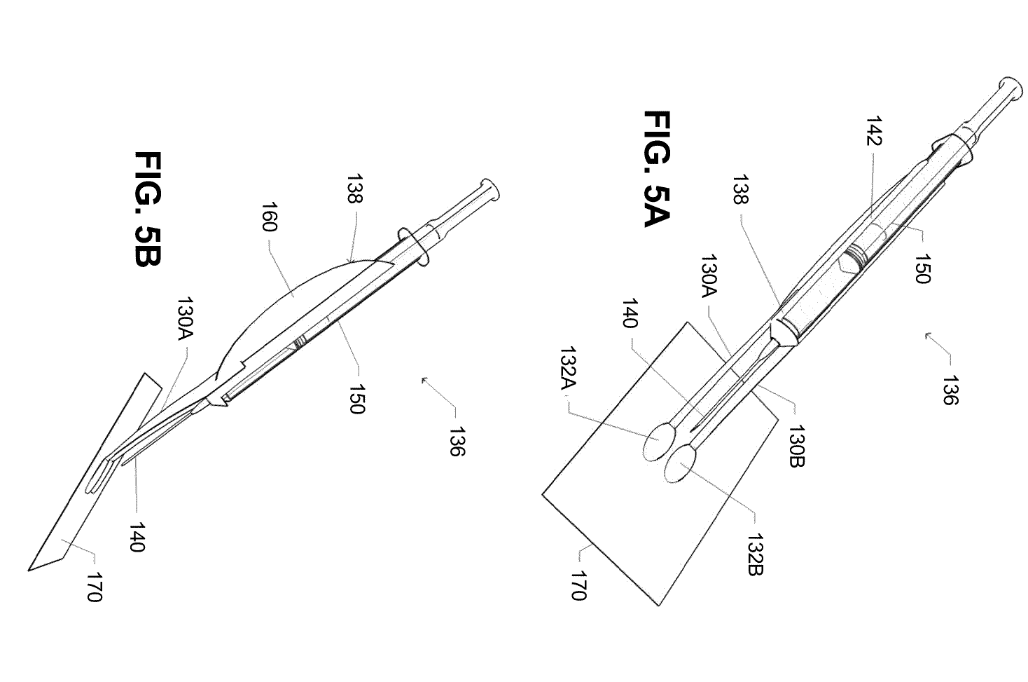

FIGS. 4A and 4B depicts a combination pain reduction and needle safety device according to another embodiment. It consists of two stimulation components 80 and 90 which fit onto the tips of the thumb 60 and forefinger 70 of either the right or the left hand. These tip covers 80 and 90 have outer layers that constitute stimulation elements 81, 91. According to one implementation, the stimulation elements 81, 91 are conductive elements that are electrically conductive throughout the entire outer surface of each element 81, 91, thereby maximizing contact against the varied curvature of the patient's skin.

Each tip cover 80, 90 also has an inner layer (such as inner layer 86 of cover 80 as best shown in FIG. 4B) which can be substantially similar to the inner layers of the embodiments discussed above.

In a further implementation, the stimulation elements 81, 91 can have features and components that are substantially similar to those described with respect to the embodiments above, including puncture resistance or a puncture resistance area 82, 92.

In this implementation as shown in FIGS. 4A and 4B, both tip covers 80 and 90 are coupled to one another by means of a flexible coupling element 122 functioning to couple the two tip covers 80 and 90 while allowing for independent movement of the two tip covers 80 and 90. A controller 120 is electrically coupled reversibly or irreversibly to both tip covers 80 and 90 via wires or similar conductive material (not shown) and can couple to the device (made up of the two fingertip elements 80, 90 and the flexible connecting element 122). The wire or other conductive material (not shown) which electrically couples the controller 120 and the two tip covers 80 and 90 is located within a hollow channel 124 defined within the flexible element 122, as best shown in FIG. 4B. The controller 120 may provide, to the stimulation components 80 and 90, electrical and/or vibration stimulation as described above. The controller 120 is comprised of a microcontroller 126, best seen in FIG. 4B, which controls various aspects of the input and the output of the controller 120 and may also function to create the TENS stimulation, vibration stimulation, or both. Like the microcontroller embodiments discussed above, this microcontroller 126 can also be configured to sense signals created by touching the tip covers 80 and 90 together in recognized patterns in order to affect various output of the microcontroller 126.

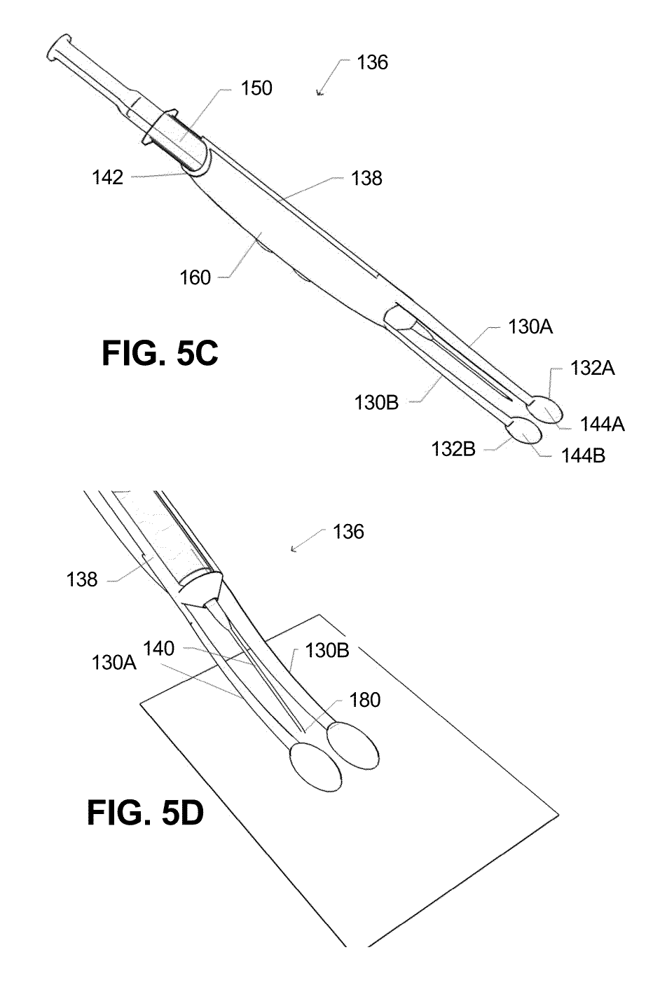

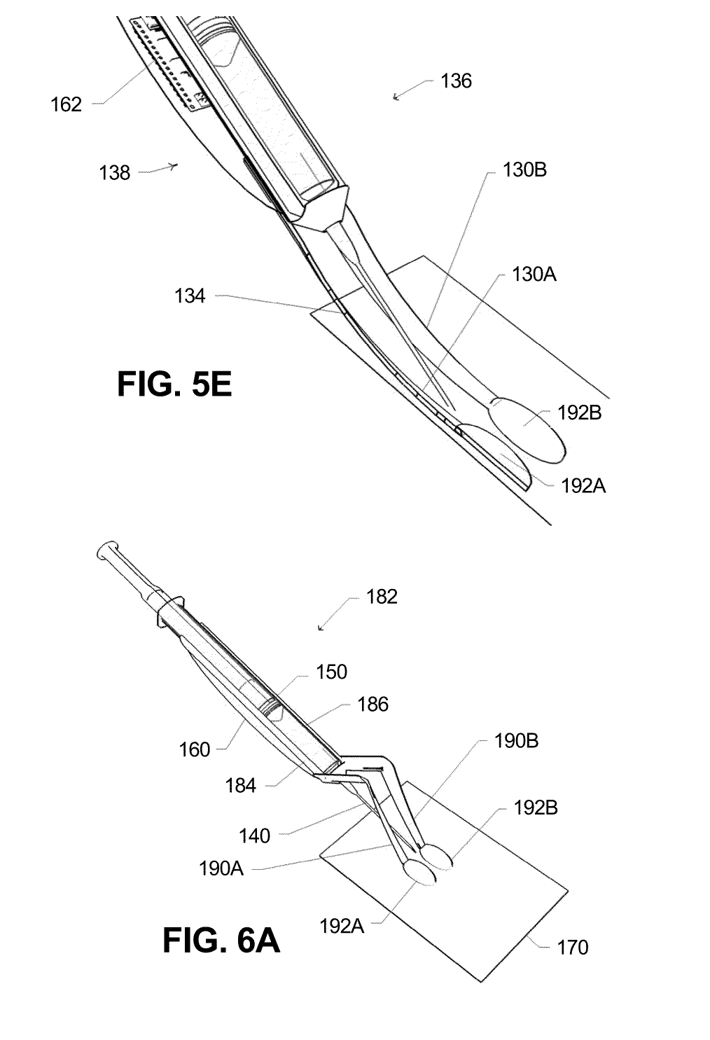

FIGS. 5A, 5B, 5C, 5D, and 5E depict another implementation of a pain reduction device 136. This device 136 has a body 138 and a pair of arms 130A, 130B coupled to a distal end of the body 138. Each arm 130A, 130B has a stimulation component 132A, 132B at a distal end of the arm 130A, 130B. The body 138 defines a syringe receiving area 142 that is shaped to receive and removably couple with a syringe typically used for cosmetic injection procedures or other types of procedures (such as the syringe 150 depicted in FIGS. 5A-5E, for example), thereby allowing the syringe (such as syringe 150) to be removably positioned within the syringe receiving area 142 such that the needle (such as exemplary needle 140) is positioned between the two arms 130A, 130B and, in certain embodiments, in proximity with the stimulation components 132A, 132B. As best shown in FIGS. 5B, 5C, and 5E, the body 138 also contains an enclosure 160 configured to contain a controller 162 (as best shown in FIG. 5E), which can include an electrical energy generating device, a vibration generating device, or both.

The controller 162 is electrically coupled via wires or similar conductive material (not shown) to the two stimulation components 132A, 132B. The wires (not shown) which electrically couple the controller 162 to the two stimulation components 132A, 132B are located within a hollow channel 134 found within each flexible arm 130A, 130B, as best shown in FIG. 5E, which shows a cutaway view of one arm 130A. It is understood that there is a similar channel defined in the other arm 130B as well. The controller 162 controls various aspects of the input and the output of the electrical energy generating component and/or the vibrational energy generating component and may also function to create the stimulation.

According to one embodiment, the device 136 can be made of typical medical grade plastic. In accordance with one specific implementation, the arms 130A, 130B can be made with a medical grade plastic with at least some degree of elasticity to allow reversible bending/flexing of the flexible arms 130A, 130B. The stimulation components 132A, 132B, in accordance with certain implementations, can have stimulation elements 144A, 144B on the underside of the stimulation components 132A, 132B (as best shown in FIG. 5C) that are configured to provide the desired stimulation (either electrical, vibrational, or both) when placed in contact with the target skin surface. In certain embodiments, the stimulation elements 144A, 144B can be constructed from electrically conductive carbonized rubber, conductive metals or similar flexible conductive material.

In an alternative implementation, instead of the device 136 having a body 138 and a pair of arms 130A, 130B, one exemplary variation can be a device that has a pair of arms and operably couples to a syringe in some fashion. That is, instead of a body that includes a syringe receiving area, the device couples to a distal end of the syringe such that the needle extending distally from the syringe is positioned between the two arms in a fashion similar to that described above with respect to the device 136.

In use, the stimulation components 132A, 132B, in one implementation, function to deliver electrical stimulation, vibrational stimulation, or both near and around the site of injection or other type of procedure in order to reduce needle pain and are shaped and positioned to allow for delivery of this stimulation when the needle 140 is injected into the skin 170 at various angles. More specifically, according to one embodiment as best shown in FIG. 5B, the flexible arms 130A, 130B are positioned such that the stimulation components 132A, 132B are disposed at a position that is proximal to the tip of the needle 140. As a result, when inserting the needle 140 into the target skin 170 for the cosmetic procedure, the configuration of the arms 130A, 130B and stimulation components 132A, 132B is such that the stimulation elements 144A, 144B must be in contact with the skin 170 in order for the needle 140 to be inserted as desired. In other words, as best shown in FIG. 5D, as the user or medical professional moves the entire device 136 distally toward the target site 180, the stimulation elements 144A, 144B make contact with the skin 170 and the arms 130A, 130B must flex (thereby increasing the contact pressure of the elements 144A, 144B on the skin 170) in order for the needle 140 to pierce the skin 170 at the site 180 and administer the treatment. Thus, the flexible arms 130A, 130B flex reversibly to allow the needle 140 to enter into the skin 170 at various angles and serves to keep the stimulation components 132A, 132B pressed onto the skin 170 during the procedure.

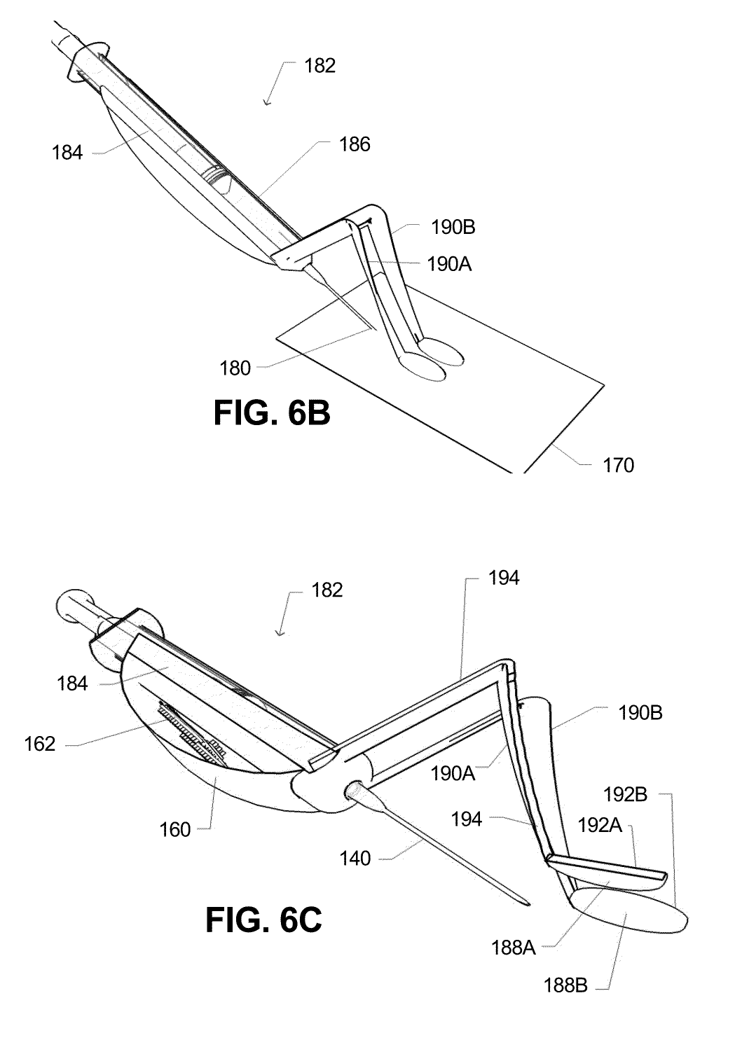

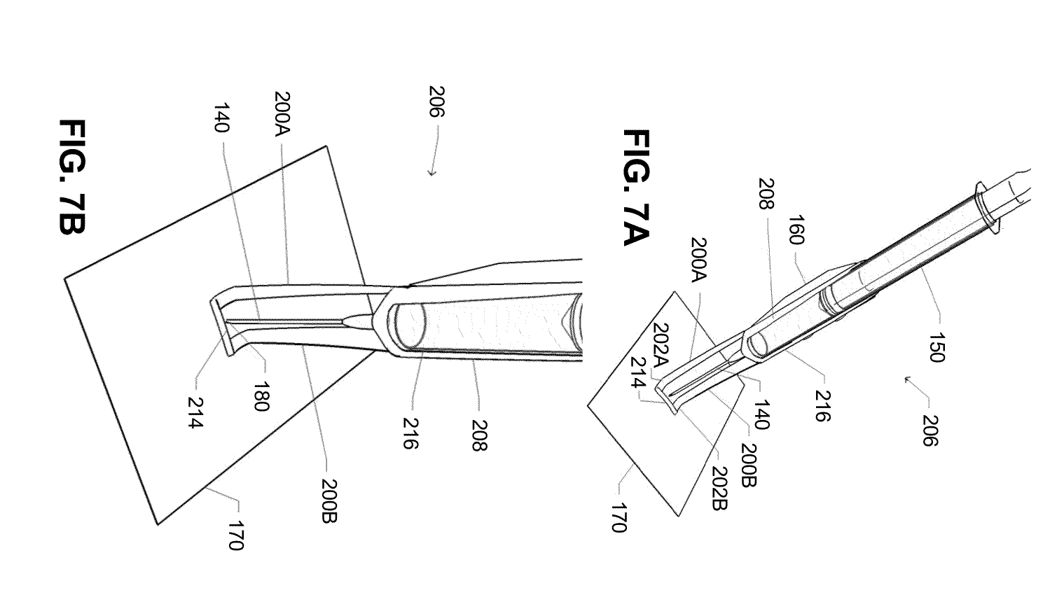

FIGS. 6A, 6B, and 6C depict a further implementation of a pain reduction device 182. This device 182 has a body 184 and a pair of arms 190A, 190B coupled to a distal end of the body 184. Each arm 190A, 190B has a stimulation component 192A, 192B at a distal end of the arm 190A, 190B. The body 184 defines a syringe receiving area 186 that is shaped to receive and removably couple with a syringe typically used for cosmetic injection procedures and other types of procedures (such as the syringe 150 depicted in FIGS. 6A-6C, for example), thereby allowing the syringe (such as syringe 150) to be removably positioned within the syringe receiving area 186 such that the needle (such as exemplary needle 140) is positioned between the two arms 190A, 190B and, in certain embodiments, in proximity with the stimulation components 192A, 192B. As best shown in FIG. 6C, the body 184 also contains an enclosure 160 configured to contain a controller 162 (as best shown in FIG. 6C), which can include an electrical energy generating device, a vibration generating device, or both.

The controller 162 is electrically coupled via wires or similar conductive material (not shown) to the two stimulation components 192A, 192B. The wires (not shown) which electrically couple the controller 162 to the two stimulation components 192A, 192B are located within a hollow channel 194 found within each flexible arm 190A, 190B (as best shown in FIG. 6C, which depicts the channel 194 in the arm 190A, with the understanding that the arm 190B has a similar channel). The controller 162 controls various aspects of the input and the output of the electrical energy generating component and/or the vibrational energy generating component and may also function to create the stimulation.

It is understood that the components and functionalities of this device 182 that are similar to those of the device 136 discussed in detail above can be made and operate in a similar fashion to those of device 136. The stimulation components 192A, 192B, according to certain embodiments, can have stimulation elements 188A, 188B on the underside of the stimulation components 192A, 192B (as best shown in FIG. 6C) that are configured to provide the desired stimulation (either electrical, vibrational, or both) when placed in contact with the target skin surface.

The flexible arms 190A, 190B in this implementation are similar to the arms 130A, 130B described above. However, these arms 190A, 190B, while still flexible, have a different configuration, with each arm 190A, 190B having a bent configuration. Regardless, these arms 190A, 190B operate in a similar fashion to the arms 130A, 130B above. That is, when inserting the needle 140 into the target skin 170 at site 180 for the cosmetic procedure, the configuration of the arms 190A, 190B and stimulation components 192A, 192B is such that the stimulation elements 188A, 188B must be in contact with the skin 170 in order for the needle 140 to be inserted as desired. In other words, as best shown in FIG. 6B, as the user or medical professional moves the entire device 182 distally toward the target site 180, the stimulation elements 188A, 188B make contact with the skin 170 and the arms 190A, 190B must flex (thereby increasing the contact pressure of the elements 188A, 188B on the skin 170) in order for the needle 140 to pierce the skin 170 at the site 180 and administer the treatment.

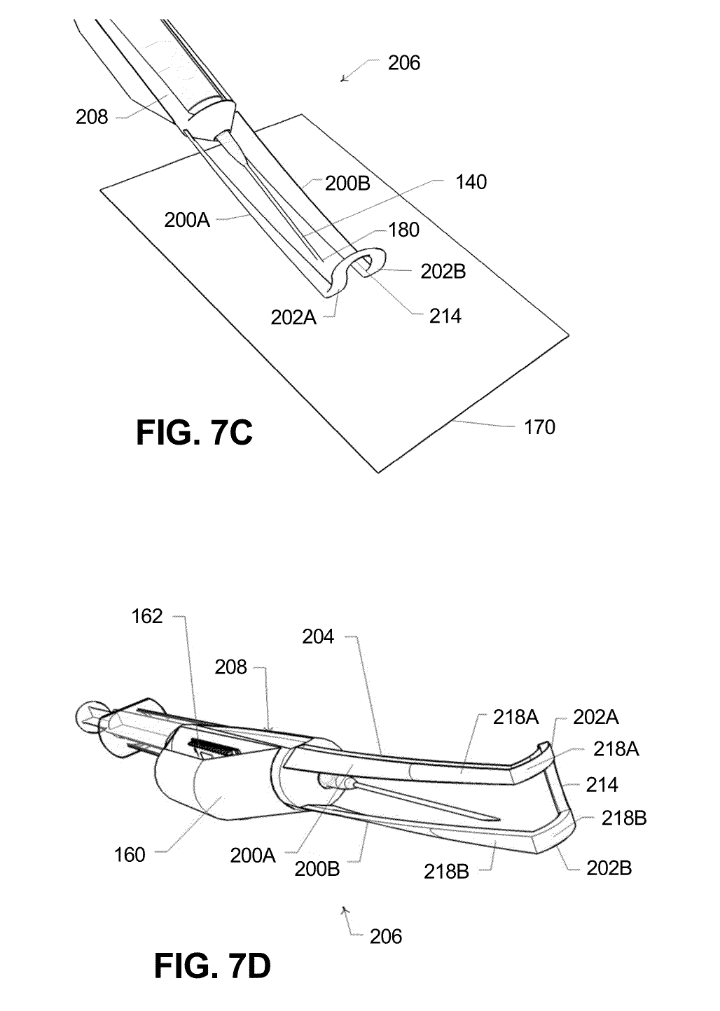

FIGS. 7A, 7B, 7C, and 7D depict a further implementation of a pain reduction device 206 according to seventh further embodiment. This device 206 has a body 208 and a pair of arms 200A, 200B coupled to a distal end of the body 208. Each arm 200A, 200B has a stimulation component 202A, 202B at a distal end of the arms 200A, 200B. The two arms 200A, 200B in this implementation are also coupled at their distal ends with a distal connection piece 214. The body 208 defines a syringe receiving area 216 that is shaped to receive and removably couple with a syringe typically used for cosmetic injection procedures and other types of procedures (such as the syringe 150 depicted in FIG. 7A, for example), thereby allowing the syringe (such as syringe 150) to be removably positioned within the syringe receiving area 216 such that the needle (such as exemplary needle 140) is positioned between the two arms 200A, 200B and, in certain embodiments, in proximity with the stimulation components 202A, 202B. As best shown in FIG. 7D, the body 208 also contains an enclosure 160 configured to contain a controller 162, which can include an electrical energy generating device, a vibration generating device, or both.

The controller 162 is electrically coupled via wires or similar conductive material (not shown) to the two stimulation components 202A, 202B. The wires (not shown) which electrically couple the controller 162 to the two stimulation components 202A, 202B are located within a hollow channel (not shown) found within each flexible arm 200A, 200B (similar to the hollow channel discussed with respect to another embodiment above and depicted in FIG. 5E). The controller 162 controls various aspects of the input and the output of the electrical energy generating component and/or the vibrational energy generating component and may also function to create the stimulation.

It is understood that the components and functionalities of this device 206 that are similar to those of the devices 136, 182 discussed in detail above can be made and operate in a similar fashion to those of devices 136, 182. The stimulation components 202A, 202B, according to certain embodiments, can have stimulation elements 218A, 218B on the underside of the stimulation components 202A, 202B (as best shown in FIGS. 7C and 7D) that are configured to provide the desired stimulation (either electrical, vibrational, or both) when placed in contact with the target skin surface.

The flexible arms 200A, 200B in this implementation are similar in some respects to the arms 130A, 130B and 190A, 190B described above. However, these arms 200A, 200B, while still flexible, have a different configuration, with the arms 200A, 200B being coupled together at the distal connection piece 214 described above. Regardless, these arms 200A, 200B operate in a similar fashion to the arms 130A, 130B and 190A, 190B above. That is, when inserting the needle 140 into the target skin 170 at the site 180 for the cosmetic procedure, the configuration of the arms 200A, 200B and stimulation components 202A, 202B is such that the stimulation elements 218A, 218B must be in contact with the skin 170 in order for the needle 140 to be inserted as desired. In other words, as best shown in FIG. 7C, as the user or medical professional moves the entire device 206 distally toward the target site 180, the stimulation elements 218A, 218B make contact with the skin 170 and the arms 200A, 200B must flex (thereby increasing the contact pressure of the elements 218A, 218B on the skin 170) in order for the needle 140 to pierce the skin 170 at the site 180 and administer the treatment.

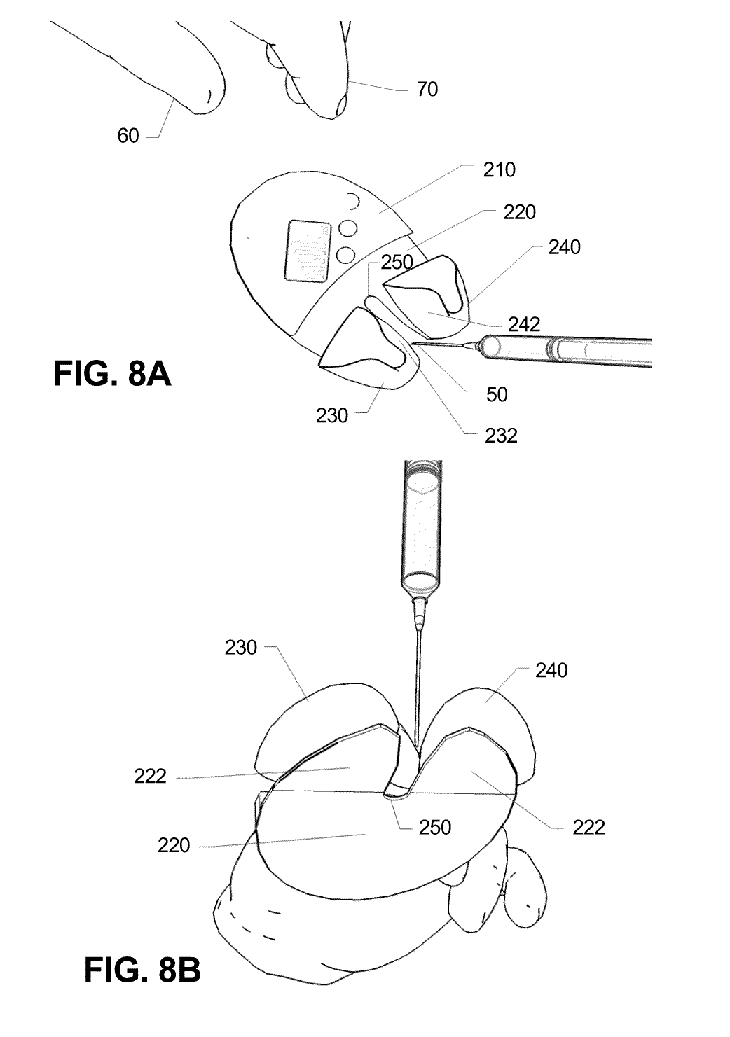

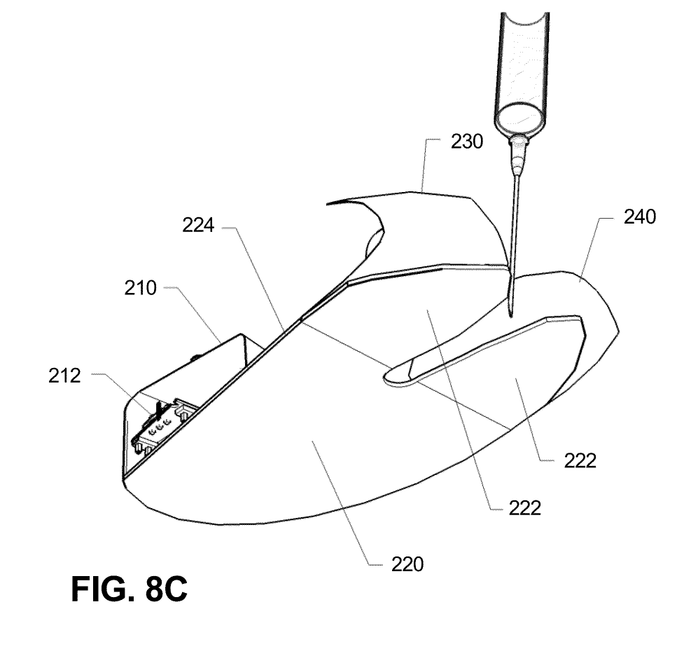

A further implementation relates to a TENS/vibration generating device which attaches to a stimulation component incorporating two stimulation elements. For example, FIGS. 8A, 8B, and 8C depict a combination pain reduction and needle safety device according to one embodiment. It consists of a controller and TENS/vibration generating device 210 which attaches reversibly or irreversibly to a specialized stimulation component 220 incorporating two electrically isolated and electrically conductive stimulation elements 222. These stimulation elements 222 are electrically coupled reversibly or irreversibly to the controller and TENS/vibration generating device 210 via wires or similar conductive material (not shown). These wires or similar conductive material may be found in a channel 224 within the specialized electrode 220, as best shown in FIG. 8C.

Attached to the stimulation component 220 are finger receiving units 230 and 240 designed to fit onto the thumb 60 and forefinger 70 of either the right or the left hand. The stimulation component 220 and stimulation elements 222 may be made of electrically conductive carbonized rubber typical of the material used to create reusable TENS electrodes as discussed above. Alternatively, they may be constructed from materials found in typical disposable TENS electrodes. The units 230 and 240 are comprised of areas 232 and 242 which are resistant to needle puncture covering the sides and tips of the thumb and forefinger and function to help prevent accidental needle stick injury. These units 230 and 240 may be made of medical grade plastic or other material able to resist needle puncture.

The controller and TENS/vibration generating component 210 may provide, to the stimulation elements 222, specialized electrical stimulation such as a randomized waveform as described above. The controller and TENS/vibration generating component 210 is comprised of a microcontroller 212, best seen in FIG. 8C which controls various aspects of the input and the output of the controller and TENS/vibration generating component 210 and may also function to create the TENS/vibration stimulation. As discussed above, this microcontroller 212 may have the ability to sense signals created by touching the stimulation elements 222 together in recognized patterns in order to affect various output of the microcontroller. A notch 250 between the two stimulation elements 222 allow for independent movement of the two elements 222 and allows for an injection or other procedure to occur near or between the two stimulation elements 222. The stimulation elements 222 serve to deliver electrical/vibration stimulation near and around the site of injection 50 (or procedure site 50) in order to reduce needle pain. The independent movement of the elements 222 allow the practitioner to freely manipulate the skin by pinching and stretching as he/she would normally do during typical cosmetic injection procedures or other types of procedures. The units 230 and 240 fit onto the thumb 60 and forefinger 70 to provide an increased level of protection against accidental needle injury.

According to additional implementations, the stimulation components described above can be incorporated into a glove that can be worn on either hand of a user. The glove device could have substantially the same components and operate in the same fashion as those described above.

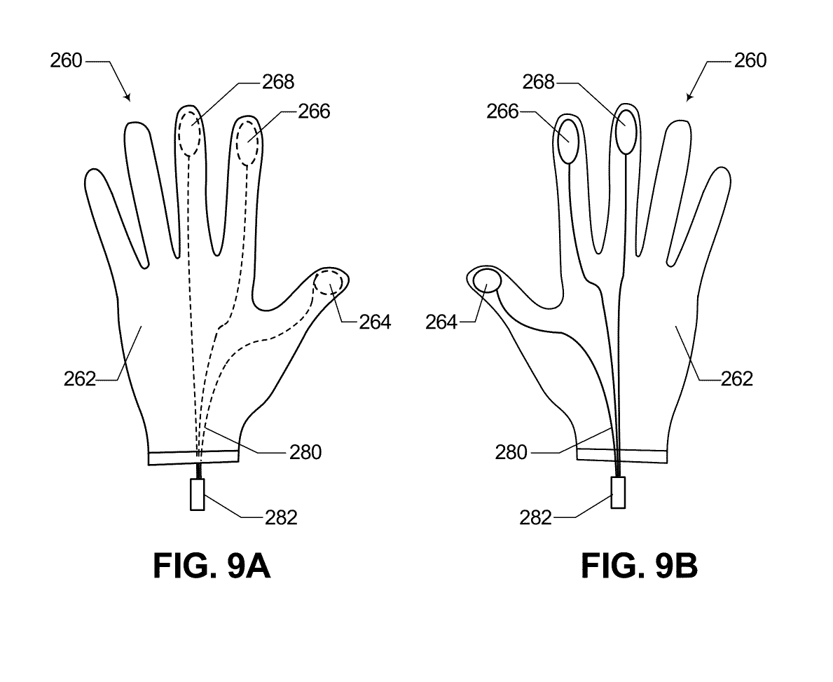

For example, FIGS. 9A and 9B depict a combination pain reduction and needle safety system 260 according to one embodiment. This system 260 is incorporated into a glove 262. FIG. 9A depicts the back of the glove 262, while FIG. 9B depicts the front or palm of the glove 262. The system 260 has three stimulation components 264, 266, 268 that are positioned on, attached to, integrated into, or otherwise associated with the fingers 270, 272, 274 of the glove 262. In this specific example as shown, the components 264, 266, 268 are positioned on the thumb 270, index finger 272, and middle finger 274 of the glove 262.

The stimulation components 264, 266, 268 are configured to be similar in form and function to the various components described above. Each component 264, 266, 268 is electrically coupled reversibly or irreversibly to one or more wires 280, which are coupled to a connector 282 associated with a controller (not shown). The controller can be incorporated into the wrist of the glove or can be a separate component that is worn on the wrist of the same hand. The controller is configured to be similar in form and function to the various controller embodiments described above.

Although the present invention has been described with reference to preferred embodiments, persons skilled in the art will recognize that changes may be made in form and detail without departing from the spirit and scope of the invention.

What is claimed is:

1. A pain reduction device for reducing the pain of injection procedures and other procedures that cause skin pain, the device comprising:
(a) a first stimulation component comprising:
(i) a first cavity defined within the first stimulation component, the first cavity configured to receive at least a portion of a first distal phalanges of a first digit of a hand of a user;
(ii) a first stimulation element disposed on an outer surface of the first stimulation component;
(b) a second stimulation component comprising:
(i) a second cavity defined within the second stimulation component, the second cavity configured to receive at least a portion of a second distal phalanges of a second digit of the hand of the user;
(ii) a second stimulation element disposed on an outer surface of the second stimulation component; and
(c) a controller operably coupled to the first and second stimulation components, the controller comprising a stimulation generating module.

2. The pain reduction device of claim 1, wherein the stimulation generating module comprises at least one of a vibration stimulation generating unit configured to transmit vibration energy to the first and second stimulation components and an electrical stimulation generating unit configured to transmit electrical energy to the first and second stimulation components.

3. The pain reduction device of claim 1, wherein the first digit comprises an index finger and the second digit comprises a thumb.

4. The pain reduction device of claim 1, wherein the first cavity is configured to receive the first distal phalanges and at least a portion of an intermediate phalanges of the first digit and the second cavity is configured to receive the second distal phalanges and at least a portion of an intermediate phalanges of the second digit.

5. The pain reduction device of claim 1, wherein at least a portion of the outer surface of the first stimulation component comprises a first substantially curved area and at least a portion of the outer surface of the second stimulation component comprises a second substantially curved area.

6. The pain reduction device of claim 5, wherein the first stimulation element is disposed on at least a portion of the first substantially curved area and the second stimulation element is disposed on at least a portion of the second substantially curved area.

7. The pain reduction device of claim 1, wherein the first stimulation component comprises a first puncture resistant area and the second stimulation component comprises a second puncture resistant area.

8. The pain reduction device of claim 1, wherein the first and second stimulation components are puncture resistant.

9. The pain reduction device of claim 1, wherein the controller is configured to detect signals created by placing the first and second stimulation components into contact with each other in predetermined patterns, wherein the controller is configured to be triggered by the signals to alter at least one operating parameter.

10. The pain reduction device of claim 1, wherein the controller is configured to be wearable on a wrist of the user.

11. The pain reduction device of claim 1, wherein the controller is configured to sense contact of at least one of the first and second stimulation elements with skin of a patient, whereby the controller is configured to be triggered to actuate the stimulation generating module to generate stimulation.

12. The pain reduction device of claim 1, wherein the pain reduction device comprises a glove, wherein the first stimulation component is associated with a first finger of the glove and the second stimulation component is associated with a second finger of the glove.

13. A pain reduction device for reducing the pain of injection procedures and other procedures that cause skin pain, the device comprising:
(a) a body comprising a syringe receiving cavity;
(b) a first flexible arm coupled to the body, the first arm extending distally from the body, the first flexible arm comprising a first stimulation component disposed at a distal end of the first flexible arm;
(c) a second flexible arm coupled to the body, the second arm extending distally from the body, the second flexible arm comprising a second stimulation component disposed at a distal end of the second flexible arm,
wherein the first and second flexible arms are coupled to the body such that a space is defined between the first and second flexible arms; and
(d) a controller operably coupled to the first and second stimulation components, the controller comprising a stimulation generating module.

14. The pain reduction device of claim 13, wherein the body is configured to removably receive a syringe in the syringe receiving cavity such that a needle extending from the syringe is positionable between the first and second flexible arms, wherein a tip of the needle is in proximity with the first and second stimulation components.

15. The pain reduction device of claim 14, wherein the first and second flexible arms are configured to flex under mild pressure, thereby allowing the needle to puncture skin in a target area while the first and second stimulation components maintain continuous contact with the skin.

16. A method for reducing the pain of injection procedures and other procedures that cause skin pain, the method comprising:
positioning a first stimulation component on a first digit of a hand of a user;
positioning a second stimulation component on a second digit of the hand;
contacting skin of a patient with the first and second stimulation components at or near a procedure site;
delivering stimulation to the skin with the first and second stimulation components; and
performing a procedure on or around the skin between or proximal to the first and second stimulation components.

17. The method of claim 16, wherein the positioning the first stimulation component on the first digit further comprises positioning the first stimulation component on a first distal phalanges of the first digit and the positioning the second stimulation component on the second digit further comprises positioning the second stimulation component on a second distal phalanges of the second digit.

18. The method of claim 16, where the delivering stimulation to the skin comprises delivering at least one of vibration stimulation and electrical stimulation.

19. The method of claim 16, wherein the delivering stimulation to the skin with the first and second stimulation components further comprises delivering the stimulation to the skin with a first stimulation element disposed on the first stimulation component and a second stimulation element disposed on the second stimulation component.

20. The method of claim 16, further comprising protecting the first and second digits from an accidental needle stick with the first and second stimulation components, wherein the first and second stimulation components are puncture resistant.
